(19) United States
(12) Patent Application Publication
(10) Pub. No.: US 2004/0265871 A1
(43) Pub. Date: Dec. 30, 2004

(54) Ultrasound stimulated DNA hybridization

(76) Inventors: Bjorn A.J. Angelsen, Trondheim (NO); Tonni F. Johansen, Trondheim (NO); Vidar Beisvag, Trondheim (NO)

Correspondence Address: Lance J. Lieberman, Esq., Cohen, Pontani, Lieberman & Pavane, Suite1210, 551 Fifth Avenue, New York, NY, 10176, US

(21) Appl. No.: 10/823,118

(22) Filed: Apr. 12, 2004

Related U.S. Application Data
(60) Provisional application No. 60/462,042, filed on Apr. 11, 2003.

(51) Int. Cl. C12Q 1/68; C12M 1/34

(52) U.S. Cl. 435/6; 435/287.2

(57) ABSTRACT

The invention composes a method and instrumentation for ultrasound stimulation of the hybridization reaction in gene expression microarray test chambers (or hybridization stations). The microarray or gene chip with the DNA molecules solution added on the surface, is mounted in a system that allows transmission of ultrasound waves into the DNA solution. The ultrasound may also be used for the washing procedure after the hybridization process

### CROSS-REFERENCE TO RELATED APPLICATION

This application claims priority from U.S. Provisional Patent Application Ser. No. 60/462,042 which was filed on Apr. 11, 2003.

### BACKGROUND OF THE INVENTION

1. Field of the Invention

The present invention is directed to laboratory equipment for analyzing gene expression and single nucleotide mutations in biological samples.

2. Description of the Related Art

The control, function, and development of cells is determined by the expression of genes from the cell nucleus. Genes are expressed as mRNA molecules which are translated into aminoacids and proteins. The so-called microarray or gene chip technology has been developed for fast, parallel identification and quantitation of multiple (−10,000) genes that are expressed in biological samples. With this technique, small droplets(spots) (diam ˜10-100 μm) containing DNA probe molecules (15-2000 bases) are placed as a grid array on a substrate (for example glass substrate) with distance ˜50 μm. mRNA is isolated from tissue or cell samples. By reverse transcription cDNA molecules are made and labelled. A solution of these “labeled” cDNAs are then added to the microarray (substrate) and hybridization (complementary base pair binding) are facilitated by incubation at ˜25-60° C. for 6-12 hours. It is also interesting to analyze solutions with fragments of genomic DNA molecules in the case of single nucleotide mutations, and in the following we refer to both the cDNA and fragmented genomic DNA molecules as DNA molecules. Through laser scanning of the microarray fluorescent signals are detected trough a photo multiplyer tube, and a digital picture is made. The fluorecente signal form each of the “spots” on the microarray are related to the expression of a specific gene in the test sample.

Albeit this method gives a parallel detection of expression of a large amount of genes, the reaction time of ˜12 h limits the throughput of the test equipment. The present invention addresses this problem by devising the use of ultrasound to stimulate the reaction speed and increase specificity.

### SUMMARY OF THE INVENTION

The invention composes a method and instrumentation for ultrasound stimulation of the hybridization reaction in gene expression microarray test chambers (or hybridization stations). The microarray or gene chip with the DNA molecules solution added on the surface, is mounted in a system that allows transmission of ultrasound waves into the DNA solution. The ultrasound may also be used for the washing procedure after the hybridization process.

The ultrasound waves effects the hybridization process in three ways:

i) With adequate intensity of the wave, the wave introduces ultrasound streaming/convection of the fluid with the DNA molecules. This increases the transportation of the DNA molecules towards the reaction sites of microarray probe DNA molecules. Without fluid convection, the transportation of the DNA molecules is produced by diffusion, which for these large molecules is a considerably slower process with subsequent slower reaction kinetics.

ii) For ultrasound frequencies in the low MHz range, the linear vibration amplitude of a typical ultrasound wave can be in the range of −1-10 nm. This is of the same order as the distance between the reaction sites between the DNA molecules and the DNA probe molecules. The ultrasound vibration hence provides fine adjustment of the molecule positions for increased reaction kinetics.

iii) Applying ultrasound in the wash-out of superfluous DNA will also help to remove the less than completely matched bindings between DNA and the the probe molecules on the arrays, hence increasing the specificity of the DNA identification and quantitation.

Based on this method, the invention further devices several methods for generation of ultrasound waves in the reaction chamber, both using ultrasound bulk wave transducers and ultrasound surface wave transducers. By driving the wave intermittently in multiple directions one can maximize the exposure of the DNA molecules of the tissue sample to the probe molecules in the arrayspots. With intermittent streaming, one can in the pauses allow diffusion of the DNA molecules over the microarry or gene chip spots for improved interaction with the DNA probe molecules.

Other objects and features of the present invention will become apparent from the following detailed description considered in conjunction with the accompanying drawings. It is to be understood, however, that the drawings are designed solely for purposes of illustration and not as a definition of the limits of the invention, for which reference should be made to the appended claims. It should be further understood that the drawings are not necessarily drawn to scale and that, unless otherwise indicated, they are merely intended to conceptually illustrate the structures and procedures described herein.

### BRIEF DESCRIPTION OF THE DRAWINGS

In the drawings:

FIG. 1 shows schematically a cross section through a hybridization reaction chamber according to the invention;

FIG. 2 shows schematically a transducer system to generate ultrasound surface waves in a non-piezoelectric ultrasound guiding plate; and

FIG. 3 shows schematically a cross section through yet another hybridization reaction chamber according to the invention.

### DETAILED DESCRIPTION OF THE PRESENTLY PREFERRED EMBODIMENTS

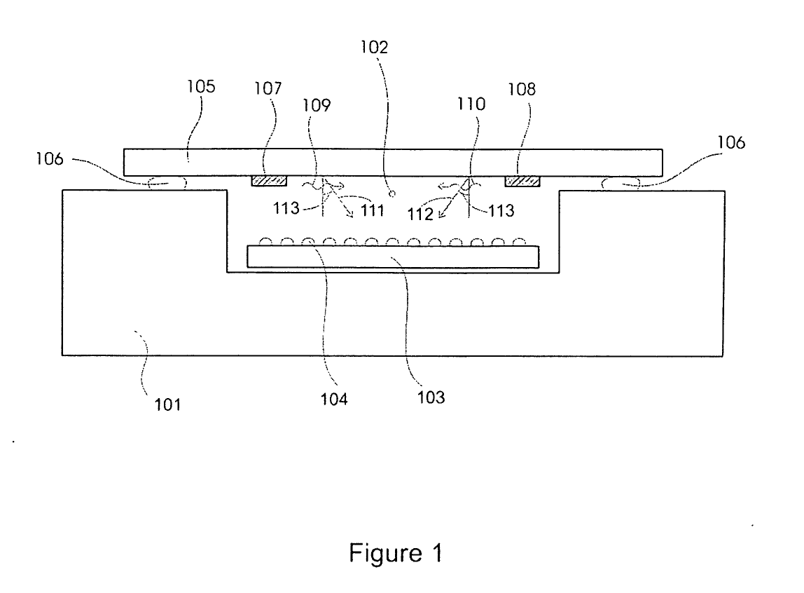

One example embodiment according to the invention is shown in a cross section in FIG. 1, where 101 shows a reaction chamber base plate with a reaction chamber 102 containing the microarray substrate 103 with the droplets 104 of DNA probe molecules. In this particular embodiment, the chamber is covered with an ultrasound guiding plate 105 and the chamber is filled with a solution of DNA molecules to be classified and quantified. Between the guiding plate and the chamber base plate could typically be a rubber gasket 106 for sealing off the reaction chamber. Such a rubber gasket will also have the desirable effect of attenuating waves and reflections at the outer edges of the ultrasound guiding plate.

On the ultrasound guiding plate 105 is in this example mounted two ultrasound transducers 107 and 108 that is connected to electric signal generators, that are not shown, so that they can excite ultrasound surface waves in the ultrasound guiding plate. The surface waves from transducer 107 is indicated by the arrow 109 and the surface wave from transducer 108 is indicated by the arrow 110. The transducers would also typically excite some waves propagating in the opposite direction, which would be attenuated by the rubber gasket 106.

In a typical operation, the surface waves excited along the ultrasound guiding plate will couple acoustic bulk waves into the fluid with propagation directions indicated by the arrows 111 and 112 for the surface waves from transducers 107 and 108, respectively. The bulk waves have a radiation angle, φ, relative to the surface normal of the guiding plate, indicated as 113. The radiation angle is determined by the ratio between the propagation velocity Cₛ of the surface wave in the guiding plate, and the propagation velocity C_{b} of the bulk wave in the fluid. From basic acoustics one can calculate the radiation angle by the formula φ=sin⁻¹(c_{b}/cₛ). The DNA solvent is usually water, which has a bulk wave propagation velocity C_{b} ˜1500 m/sec, where the surface wave propagation velocity of the ultrasound guiding plate can vary from cₛ-1700 m/s (Pt) to cₛ ˜6040 m/s (Al₂O₃), or even higher for other ceramics and especially Beryllium. Hence by selection of the material in the ultrasound guiding plate one can vary the bulk wave radiation angle in a range from −15-65 deg.

The power absorption of the bulk wave in the fluid will generate a streaming force along the propagation direction of the bulk wave. The streaming force will subsequently produce a convection of the fluid which will improve the transportation of the DNA molecules in the fluid towards the probe DNA molecules on the substrate, hence increasing the reaction speed. To impose complex stirring of the DNA molecules in front of the substrate, one could typically in a time sequence switch the bulk wave directions sequentially by switching between different driving transducers, for example transducer 107 and 108 of FIG. 1. Additional transducers driving waves with propagation directions with components normal to the drawing section could also be used. Simultaneous driving of the transducers would introduce further complex stirring forces of the DNA molecule solution.

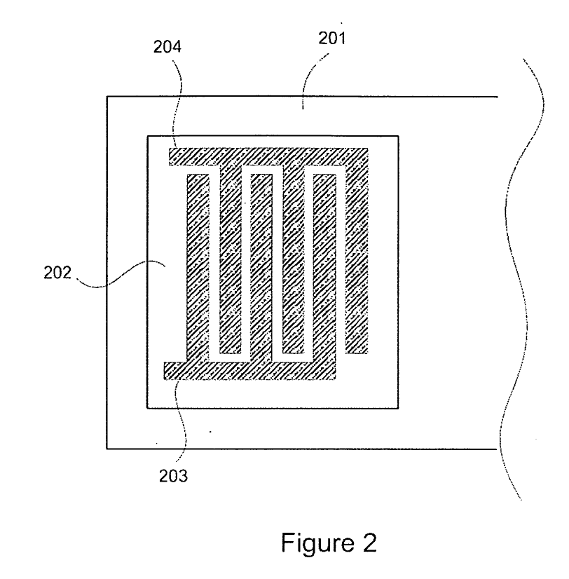

Surface wave ultrasound transducers could be based on a piezoelectric ceramic film on a substrate coated with metal electrodes in a finger pattern as illustrated in FIG. 2. In this Figure, 201 shows part of the ultrasound guiding plate covered with a piezoelectric, ceramic film 202 that is further covered with a pair of finger electrodes 203 and 204 of electrically conducting material. The ceramic film with the finger electrodes constitute one of the transducers 107 or 108 in FIG. 1. Introducing an oscillating voltage between the finger electrodes produces compressions and elongations of the piezoelectric film along the surface of the guiding plate 201, according to known methods, which generates the ultrasound surface wave.

A low cost material for the ultrasound guiding plate 201 is alumina (Al₂O₃), which would give a bulk wave radiation angle into the water solution of φ˜15 deg. Such a guiding plate with a printed piezoelectric film would give low manufacturing cost. A guiding plate composed of platinum would give a radiation angle φ˜60 deg, at a somewhat higher cost. Another interesting material for the guiding plate is a fully piezoelectric ceramic plate, where the conducting finger electrodes can be attached directly to the plate. Ceramic piezoelectric materials have surface acoustic velocities cₛ ˜2400 m/s which gives a bulk wave radiation angle φ˜40 deg. Other materials are also interesting, but not listed here, as particular material selection is obvious within the scope of the invention.

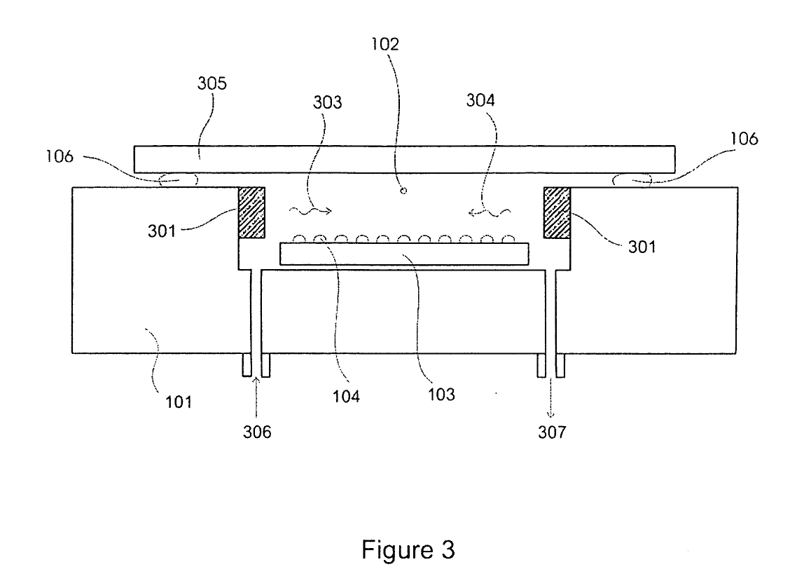

Another method to generate ultrasound bulk waves in the fluid is with direct bulk wave transducers, as illustrated in a cross section FIG. 3. This Figure shows a modification of FIG. 1, with the difference that the top plate 305 no longer contains ultrasound transducers. The ultrasound bulk waves in the DNA solution are in this embodiment according to the invention generated by separate bulk wave transducers, where this Figure shows by way of example two bulk wave transducers 301 and 302 for transmitting bulk waves with propagation directions indicated by the arrows 303 and 304, respectively. The material in the top plate 305 of the reaction chamber 102 can now be selected with less restrictions, as it is not transporting a surface wave that is used to generate bulk waves into the reaction chamber. With this particular positioning of the transducers, the bulk waves would propagate along the substrate 103, producing a streaming force in its propagation direction. Vertical convection of the solution would be introduced by the physical limitations of the reaction chamber which do not allow extended horizontal fluid convection only. The bulk wave transducers could be made of conventional piezoceramic materials or piezoceramic films, or as capacitive micromachined ultrasound transducers on Silicon, socalled cmuts

Ultrasound waves are also useful in the washing process of the microarray or gene chip after the hybridization process. One embodiment for such washing according to the invention, is illustrated in FIG. 3, where 306 shows an inlet of a cleaning fluid to the reaction chamber, with 307 as an outlet. The inlet and the outlet is connected to a fluid/pumping system according to known methods. After the hybridization process, washing fluid is pumped through the reaction chamber 102, while the ultrasound transducers 301 and 302 are activated to transmit ultrasound waves onto the array surface to facilitate removal of all components of DNA solution from the array surface.

In other embodiments, the micro array substrate, 103, could conveniently be mounted in direct contact with the ultrasound transducer, so that ultrasound vibrations were generated directly in the substrate and coupled into the DNA solution. The ultrasound transducers can be of the bulk wave type, as illustrated in FIG. 3, or of the surface wave type with coupling into bulk waves, as illustrated in FIGS. 1 and 2. In this last example, the micro array substrate can be mounted onto a plate with attached ultrasound surface wave transducers similar to the ones illustrated in FIG. 2. Such ultrasound transducers could also be mounted directly onto the micro array substrate 103. Such direct mounting would require that the transducers can be manufactured at low cost, as is the case with thick film printing of ceramic films onto the substrate as described in relation to FIG. 2. In all these situations, the ultrasound transducers can also be used for cleaning of the micro array after the hybridization process.

The ultrasound bulk waves in the DNA solution, and for cleaning of the microarray or gene chip after the hybridization process, could also be generated with transducers that are totally outside the reaction chamber. This could be transducers that are in direct contact with the reaction chamber, or the reaction chamber could be immersed in a fluid where the ultrasound is transmitted via this fluid into the reaction chamber. In this last situation, one could immerse several reaction chambers in the fluid for processing of many micro-arrays in parallel.

Thus, while there have shown and described and pointed out fundamental novel features of the invention as applied to a preferred embodiment thereof, it will be understood that various omissions and substitutions and changes in the form and details of the devices illustrated, and in their operation, may be made by those skilled in the art without departing from the spirit of the invention. It is also expressly intended that all combinations of those elements and/or method steps which perform substantially the same function in substantially the same way to achieve the same results are within the scope of the invention. Moreover, it should be recognized that structures and/or elements and/or method steps shown and/or described in connection with any disclosed form or embodiment of the invention may be incorporated in any other disclosed or described or suggested form or embodiment as a general matter of design choice. It is the intention, therefore, to be limited only as indicated by the scope of the claims appended hereto.

We claim:

1. A method for increasing the reaction velocity of chemical binding of DNA to DNA probe molecules in a microarray or gene chip system for identification and quantitation of gene expression or single nuclotide mutations, where the solution that contains the DNA molecules is insonified with ultrasound.

2. A method according to claim 1, where the ultrasound waves produce streaming in the DNA solution.

3. A method for increasing the processing speed of DNA binding to DNA-probe molecules on a micro array, where ultrasound waves are used in the washing process of the micro array after the hybridization process.

4. A method according to claim 1, where the ultrasound is generated by bulk wave transducers in acoustic contact with the DNA solution.

5. A method according to claim 1, where the ultrasound bulk waves in the DNA are generated from ultrasound surface waves in a material in contact with the DNA solution.

6. A method according to claim 5, where the ultrasound surface waves are generated by electromechanical coupling between a piezoceramic film on the surface of said material in contact with the DNA solution, and metallic finger electrodes on the surface of said piezoeramic film.

7. A method according to claim 1, where the ultrasound bulk waves in the DNA solution are generated with cmut ultrasound transducers.

8. A method according claim 5, where said material in contact with the DNA solution is the micro array substrate itself.

9. A method according to claim 4, where the micro array substrate is mounted directly onto said bulk wave transducers.

10. A method according to claim 9, where said bulk wave transducers are made as piezoceramic films adhered to the micro array substrate.

11. A method according to claim 1, where the ultrasound is transmitted from the transducers that are external to the reaction chamber, the transducers being either in direct contact with the reaction chamber or in acoustic contact with the reaction chamber through a contact material, such as a fluid or a solid.

12. A method according to claim 11, where several micro-array reaction chambers are processed in parallel, where all the reaction chambers are in contact with the same material where the ultrasound waves are generated, wherefrom the ultrasound waves are coupled into all reaction chambers in parallel.

13. A method according to claim 3, where the ultrasound is generated by bulk wave transducers in acoustic contact with the DNA solution.

14. A method according to claim 3, where the ultrasound bulk waves in the DNA are generated from ultrasound surface waves in a material in contact with the DNA solution.

15. A method according to claim 3, where the ultrasound bulk waves in the DNA solution are generated with cmut ultrasound transducers.

16. A method according to claim 3, where the ultrasound is transmitted from the transducers that are external to the reaction chamber, the transducers being either in direct contact with the reaction chamber or in acoustic contact with the reaction chamber through a contact material, such as a fluid or a solid.
